(19) United States
(12) Patent Application Publication
(10) Pub. No.: US 2026/0306096 A1
(43) Pub. Date: Oct. 8, 2026

(54) POWERED STAPLING DEVICE WITH MANUAL OVERRIDE MECHANISM

(71) Applicant: Covidien LP, Mansfield, MA (US)

(72) Inventors: Xingrui CHEN, Glastonbury, CT (US); David A. NICHOLAS, Trumbull, CT (US); Patrick GUTELIUS, Monroe, CT (US); Brian J. CRESTON, Madison, CT (US); Fabio A. PINTO, Stamford, CT (US)

(21) Appl. No.: 19/478,959

(22) PCT Filed: Apr. 25, 2024
(86) PCT No.: PCT/IB2024/054045

§ 371 Date: Oct. 27, 2025

Related U.S. Application Data
(60) Provisional application No. 63/462,624, filed on Apr. 28, 2023.

(51) Int. Cl. A61B 17/068; A61B 17/00

(52) U.S. Cl. CPC A61B 17/068; A61B 2017/00398; A61B 2017/00407

(57) ABSTRACT

A surgical device includes a powered handle assembly that is coupled to a tool assembly by an elongate body. The powered handle assembly includes a motor assembly that is coupled to a toothed rack by a gear assembly. The motor assembly can be actuated to advance or retract the toothed rack to actuate the tool assembly. The handle assembly includes a housing that defines an opening that is dimensioned to receive a retraction tool that is configured to engage the gear assembly and manually override the motor assembly to facilitate manual retraction of the toothed rack. The retraction tool is configured to effect only retraction of the toothed rack.

This Application claims priority from U.S. Provisional Patent Application 63/462,624, filed 28 Apr. 2023, the entire content of which is incorporated herein by reference.

### FIELD

This disclosure is directed to powered surgical devices and, more particularly, to powered surgical devices with manual override mechanisms.

### BACKGROUND

Various types of surgical devices used to endoscopically treat tissue are known in the art, and are commonly used, for example, for closure of tissue or organs in transection, resection, and anastomoses procedures, for occlusion of organs in thoracic and abdominal procedures, and for electrosurgically fusing or sealing tissue.

One example of such a surgical device is a surgical stapling device. Typically, surgical stapling devices include a tool assembly and a drive assembly. The tool assembly includes an anvil assembly and a cartridge assembly, and the drive assembly is movable through the tool assembly to actuate the tool assembly, i.e., to approximate the cartridge and anvil assemblies and to advance an actuation sled through the cartridge assembly to eject staples from the cartridge assembly. In some surgical stapling devices, the drive assembly includes a flexible drive beam and a clamp member that is supported on a distal end of the drive beam.

Surgical stapling devices can be manually actuated devices in which a clinician squeezes a trigger to actuate the stapling device, or powered stapling devices in which a clinician activates a motor within the stapling device to actuate the stapling device. Although powered stapling devices require less force to operate, difficulties may arise when the device loses power or components of the device malfunction or become damaged. In such instances, the device remains clamped about tissue preventing removal of the device from a patient.

A continuing need exists in the art for a powered stapling device that includes a drive assembly that can be manually retracted when power is lost or when the device is not operational to facilitate removal from a patient.

### SUMMARY

A surgical device includes a powered handle assembly that is coupled to a tool assembly by an elongate body. The powered handle assembly includes a motor assembly that is coupled to a toothed rack by a gear assembly. The motor assembly can be actuated to advance or retract the toothed rack to actuate the tool assembly. The handle assembly includes a housing that defines an opening that receives a retraction tool that is configured to engage the gear assembly and manually override the motor assembly to facilitate manual retraction of the toothed rack. The retraction tool may be configured to effect only retraction of the toothed rack.

Aspects of the disclosure are directed to a powered handle assembly for a surgical device that includes a housing, a motor assembly, a toothed rack, a gear shaft, a first gear, a second gear, and a retraction tool. The housing defines a cavity and an access opening that communicates with the cavity. The motor assembly is supported within the cavity of the housing and includes a motor shaft and an output gear that is secured to the motor shaft. The toothed rack is received within the cavity of the housing and is supported for longitudinal movement between a rack retracted position and a rack advanced position. The gear shaft is supported within the cavity of the housing. The first gear is rotatably supported on the gear shaft and is engaged with the output gear to translate rotational movement of the output gear to rotation of the first gear. The second gear is rotatably supported on the gear shaft and is engaged with the toothed rack such that rotation of the second gear causes longitudinal movement of the toothed rack. The second gear is linearly movable along the gear shaft from a first position in which the second gear is coupled to the first gear such that rotation of the first gear causes rotation of the second gear, to a second position in which the second gear can rotate independently of the first gear. The retraction tool includes a handle and an operating member. The operating member is insertable through the access opening in the housing into engagement with the second gear to an engaged position, wherein in the engaged position, the second gear is moved from the first position to the second position and the operating member is engaged with the second gear such that rotation of the operating member causes rotation of the second gear.

Still other aspects of the disclosure are directed to a surgical stapling device including a handle assembly, an adapter assembly, a firing rod, and a tool assembly. The handle assembly includes a housing, a motor assembly, a toothed rack, a gear shaft, a first gear, a second gear, and a retraction tool. The housing defines a cavity and an access opening that communicates with the cavity. The motor assembly is supported within the cavity of the housing and includes a motor shaft and an output gear that is secured to the motor shaft. The toothed rack is received within the cavity of the housing and is supported for longitudinal movement between a rack retracted position and a rack advanced position. The gear shaft is supported within the cavity of the housing. The first gear is rotatably supported on the gear shaft and is engaged with the output gear to translate rotational movement of the output gear to rotation of the first gear. The second gear is rotatably supported on the gear shaft and is engaged with the toothed rack such that rotation of the second gear causes longitudinal movement of the toothed rack. The second gear is linearly movable along the gear shaft from a first position in which the second gear is coupled to the first gear such that rotation of the first gear causes rotation of the second gear, to a second position in which the second gear can rotate independently of the first gear. The retraction tool includes a handle and an operating member. The operating member is insertable through the access opening in the housing into engagement with the second gear to an engaged position, wherein in the engaged position, the second gear is moved from the first position to the second position and the operating member is engaged with the second gear such that rotation of the operating member causes rotation of the second gear. The adapter assembly has a proximal portion and a distal portion, and the proximal portion is coupled to the handle assembly. The firing rod has a proximal portion that is coupled to the toothed rack and a distal portion. The tool assembly is supported on the distal portion of the adapter assembly and is operatively associated with the firing member.

In aspects of the disclosure, the powered handle assembly includes a retainer assembly that is supported within the housing and is configured to engage the second gear to retain the second gear in the second position.

In some aspects of the disclosure, the second gear includes a first end portion that includes protrusions, the first gear includes a central hub that defines a through bore, and an inner surface of the central hub defines recesses that receive the protrusions of the second gear when the second gear is in the first position.

In certain aspects of the disclosure, the second gear includes a second end portion that defines an annular groove, and the retainer assembly includes a gear clip that has resilient legs that are received within the annular groove when the second gear is in the second position to retain the second gear in the second position.

In aspects of the disclosure, the second end portion of the second gear includes a tapered surface that biases the resilient legs outwardly to allow the resilient legs to move into the annular groove as the second gear moves from the first position to the second position.

In some aspects of the disclosure, the operating member of the retraction tool includes first castellations and the first end portion of the second gear includes second castellations, and the first castellations mesh with the second castellations to couple the operating member of the retraction tool to the second gear such that rotation of the operating member of the retraction tool causes rotation of the second gear.

In certain aspects of the disclosure, the retraction tool includes a handle and the operating member, and the handle is coupled to the operating member such that rotation of the handle in a first direction causes corresponding rotation of the operating member and rotation of the handle in a second opposite direction causes the handle to disengage from the operating member.

In aspects of the disclosure, the handle has a T-shaped configuration and defines a slot having an open side and a circular bore, and the circular bore defines a longitudinal axis that is perpendicular to a longitudinal axis defined by the slot.

In some aspects of the disclosure, the operating member is L-shaped and includes a first portion that is received within the circular bore and a second portion that is received within the slot such that rotation of the handle of the retraction tool in the second opposite direction causes the operating member to exit from the open side of the slot.

In certain aspects of the disclosure, rotation of the handle in the first direction causes the toothed rack to move from a rack advanced position to a rack retracted position.

Other aspects of the disclosure are directed to a retraction tool including a handle and an operating member. The handle has a T-shaped configuration and defines a slot having an open side and a circular bore. The circular bore defines a longitudinal axis that is perpendicular to a longitudinal axis defined by the slot. The operating member has an L-shaped configuration and includes a first portion that is received within the circular bore and a second portion that is received within the slot. Rotation of the handle in a first direction causes corresponding rotation of the operating member and rotation of the handle in a second opposite direction causes the second portion of the operating member to exit from the slot via the open side of the slot.

Other aspects of the disclosure are directed to a powered handle assembly for a surgical device that includes a housing, a motor assembly, a toothed rack, a gear shaft, a first gear, and a second gear. The housing defines a cavity and an access opening that communicates with the cavity. The motor assembly is supported within the cavity of the housing and includes a motor shaft and an output gear secured to the motor shaft. The toothed rack is received within the cavity of the housing and is supported for longitudinal movement between a rack retracted position and a rack advanced position. The gear shaft is supported within the cavity of the housing, has a first end portion and a second end portion, and movable between first and second positions. The first gear is rotatably supported on the gear shaft and is engaged with the output gear to translate rotational movement of the output gear to rotation of the first gear. The first gear is engaged with the first end portion of the gear shaft when the gear shaft is in the first position to translate rotational movement of the first gear to rotational movement of the gear shaft. The first gear is disengaged from the first end portion of the gear shaft when the gear shaft is in the second position to allow the gear shaft to rotate independently of the first gear. The second gear is secured to the gear shaft and is engaged with the toothed rack such that rotation of the second gear causes longitudinal movement of the toothed rack. The retraction tool includes a handle and an operating member that is insertable through the access opening in the housing into engagement with the second end portion of the gear shaft to move the gear shaft from the first position to the second position.

In aspects of the disclosure, the second end portion of the gear shaft includes screw threads, and the operating member of the retraction tool defines an internal bore and includes internal screw threads positioned about the internal bore.

In some aspects of the disclosure, the second end portion of the gear shaft is received within the internal bore of the operating member of the retraction tool when the operating member is inserted through the access opening, such that rotation of the retraction tool in a first direction draws the second end portion of the gear shaft into the internal bore of the operating member of the retraction tool to move the gear shaft from the first position to the second position.

In certain aspects of the disclosure, the retraction tool includes resilient fingers that are positioned about the operating member, and each of the resilient fingers has a tapered end and a shoulder.

In aspects of the disclosure, the tapered ends facilitate passage of the resilient fingers through the access opening, and the shoulders engage the housing when the operating member is inserted through the access opening to secure the operating member within the access opening.

In some aspects of the disclosure, rotation of the retraction tool in a second direction opposite to the first direction causes the operating member to disengage from the second end portion of the gear shaft.

Other features of the disclosure will be appreciated from the following description.

### BRIEF DESCRIPTION OF THE DRAWINGS

Various aspects of the disclosed surgical device are described herein below with reference to the drawings, wherein:

FIG. 1 is a side perspective view of a surgical device and a retraction tool according to aspects of the disclosure with a tool assembly of the surgical device in a clamped position and the retraction tool separated from the surgical device;

FIG. 2 is a side perspective view from a first side of a handle assembly of the surgical device shown in FIG. 1 with a housing section of the handle assembly removed to expose internal components of the handle assembly including a motor assembly, a toothed rack, a gear assembly, and a bevel gear;

FIG. 2A is a side perspective view of a second gear of the gear assembly shown in FIG. 2;

FIG. 3 is a side perspective view from a second side of the handle assembly shown in FIG. 2 with another housing section of the handle assembly removed to expose the internal components of the handle assembly including the motor assembly, the toothed rack, the gear assembly, and the bevel gear;

FIG. 4 is a side perspective exploded view of components of the handle assembly including the motor assembly, the toothed rack, the gear assembly, and the bevel gear;

FIG. 5 is a side perspective view of a spur gear of the mechanism and the bevel gear shown in FIG. 4 with parts separated;

FIG. 6 is a perspective view from one end of a retaining assembly of the handle assembly shown in FIG. 4;

FIG. 7 is a cross-sectional view taken along section line 7 -7 of FIG. 2;

FIG. 8 is a cross-sectional view taken along section line 8 -8 of FIG. 3;

FIG. 9 is a side perspective view of the retraction tool and the spur gear of the surgical device shown in FIG. 1 with the retraction tool separated from the spur gear;

FIG. 10 is a cross-sectional view taken through the retraction tool and the handle assembly of the surgical device shown in FIG. 1 with the retraction tool engaged with the spur gear of the handle assembly and the spur gear engaged with the bevel gear;

FIG. 11 is a side perspective view of the retraction tool and the spur gear shown in FIG. 9 with the retraction tool engaged with the spur gear;

FIG. 12 is a cross-sectional view taken through the retraction tool and the handle assembly of the surgical device shown in FIG. 1 with the retraction tool engaged with the spur gear of the handle assembly and the spur gear disengaged from the bevel gear;

FIG. 13 is a cross-sectional view taken along section line 13 -13 of FIG. 12;

FIG. 14 is a side perspective view from the first side of the handle assembly of the surgical device shown in FIG. 1 with the housing section of the handle assembly removed and the retraction tool inserted into the surgical device as the retraction tool is rotated to retract the rack of the handle assembly;

FIG. 15 is a side perspective view of the handle assembly of the surgical device shown in FIG. 1 with the retraction tool inserted into the surgical device as the retraction tool is rotated to retract the rack of the handle assembly;

FIG. 16 is a side perspective exploded view of an alternate version of internal components of the handle assembly of the surgical device shown in FIG. 1;

FIG. 17 is a perspective view from one end of an alternate version of the retraction tool shown in FIG. 1;

FIG. 18 is a side perspective view of the retraction tool shown in FIG. 18;

FIG. 19 is a cross-sectional view taken through the handle assembly including the internal components of the handle assembly shown in FIG. 16;

FIG. 20 is a cross-sectional view taken through the handle assembly as shown in FIG. 19 with the retraction tool partially inserted into the handle assembly;

FIG. 21 is a cross-sectional view taken through the handle assembly as shown in FIG. 20 with the retraction tool fully inserted into the handle assembly;

FIG. 22 is a cross-sectional view taken through the handle assembly as shown in FIG. 21 with the retraction tool fully inserted into the handle assembly as the retraction tool is rotated to partially retract a gear shaft of the gear assembly;

FIG. 23 is a cross-sectional view taken through the handle assembly as shown in FIG. 22 with the retraction tool fully inserted into the handle assembly as the retraction tool is rotated to further retract the gear shaft of the gear assembly;

FIG. 24 is a cross-sectional view taken through the handle assembly as shown in FIG. 23 with the retraction tool fully inserted into the handle assembly as the retraction tool is rotated to fully retract the gear shaft of the gear assembly; and FIG. 25 is a side perspective view of the handle assembly shown in FIG. 1 including the internal components of the handle assembly shown in FIG. 16 as the retraction tool is rotated to retract the rack of the handle assembly.

### DETAILED DESCRIPTION

The disclosed surgical device will now be described in detail with reference to the drawings in which like reference numerals designate identical or corresponding elements in each of the several views. However, it is to be understood that aspects of the disclosure are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the device in its customary fashion, while the term “distal” is used generally to refer to that portion of the device that is farther from the clinician during use of the device in its customary fashion. In addition, directional terms such as front, rear, upper, lower, top, bottom, and similar terms are used to assist in understanding the description and are not intended to limit the disclosure. Further, the term “clinician” is used generally to refer to medical personnel including doctors, nurses, surgeons, and support personnel.

This disclosure is directed to a surgical device that includes a powered handle assembly that is coupled to a tool assembly by an adapter assembly. The powered handle assembly includes a motor assembly that is coupled to a toothed rack by a gear assembly. The motor assembly can be actuated to advance or retract the toothed rack to actuate the tool assembly. The handle assembly includes a housing that defines an access port that receives a retraction tool that is configured to engage the gear assembly and manually override the motor assembly to facilitate manual retraction of the toothed rack. The retraction tool may be configured to effect only retraction of the toothed rack.

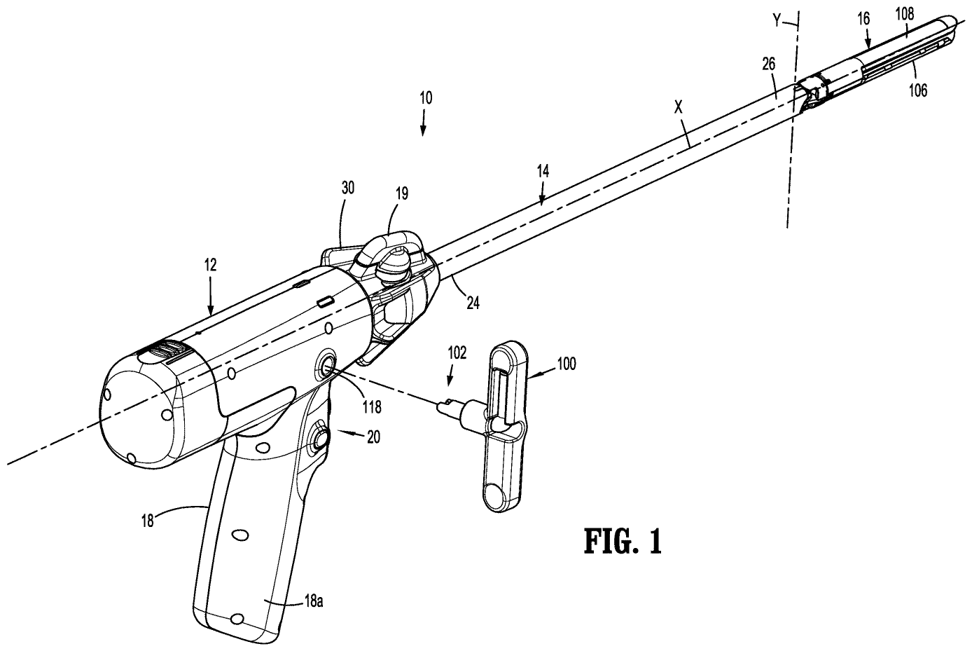

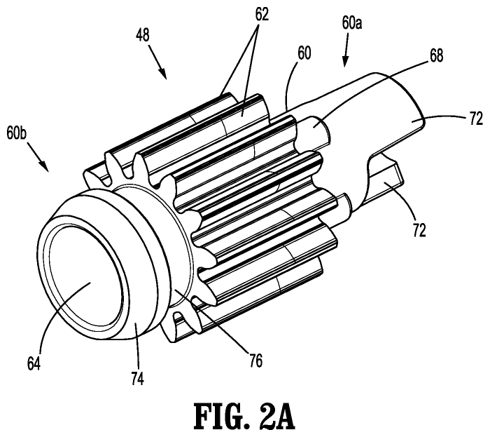

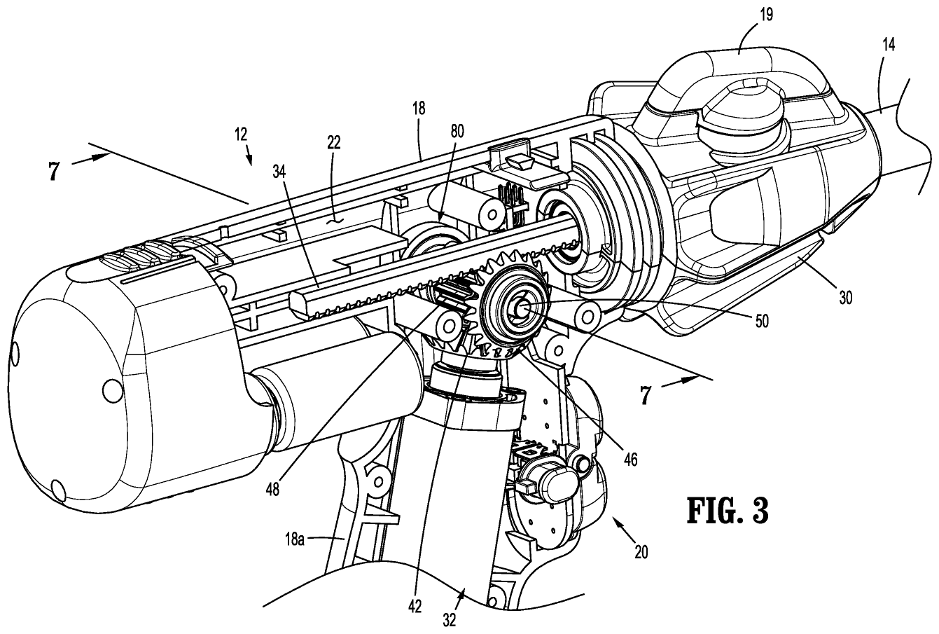

FIG. 1 illustrates a surgical device shown generally as stapling device 10 that includes a handle assembly 12, an elongate body 14, and a tool assembly 16. The handle assembly 12 includes a housing 18 that forms a stationary handle portion 18a, an articulation lever 19, and an actuation button or buttons 20. The actuation buttons 20 control operation of different mechanisms of the stapling device 10 to effect clamping and firing of the stapling device 10. In aspects of the disclosure, the housing 18 of the handle assembly 12 is formed from half-sections that are coupled together such as by welding or with screws to define a cavity 22 (FIG. 3) that receives internal components of the handle assembly 12 which are described in further detail below.

The adapter assembly 14 defines a longitudinal axis “X” and includes a proximal portion 24 that is coupled to the handle assembly 12 and a distal portion 26 that supports the tool assembly 16. In aspects of the disclosure, the tool assembly 16 is secured to the distal portion 26 of the elongate body 14 by a pivot member (not shown) that defines an articulation axis “Y” that is transverse to the longitudinal axis “X”. The articulation lever 19 is operatively coupled to the tool assembly 16 via an articulation linkage (not shown) such that manipulation of the articulation lever 19 causes articulation of the tool assembly 16 about the articulation axis “Y” between a non-articulated position in which the tool assembly 16 defines a longitudinal axis that is aligned with the longitudinal axis “X” and non-articulated positions in which a longitudinal axis of the tool assembly 16 and the longitudinal axis “X” of the elongate body 14 define acute angles. The proximal portion 24 of the adapter assembly 14 is supported within a rotation knob 30 that is rotatably coupled to a distal portion of the handle assembly 12. The rotation knob 30 is manually rotatable about the longitudinal axis “X” to rotate the elongate body 14 and the tool assembly 16 about the longitudinal axis “X”.

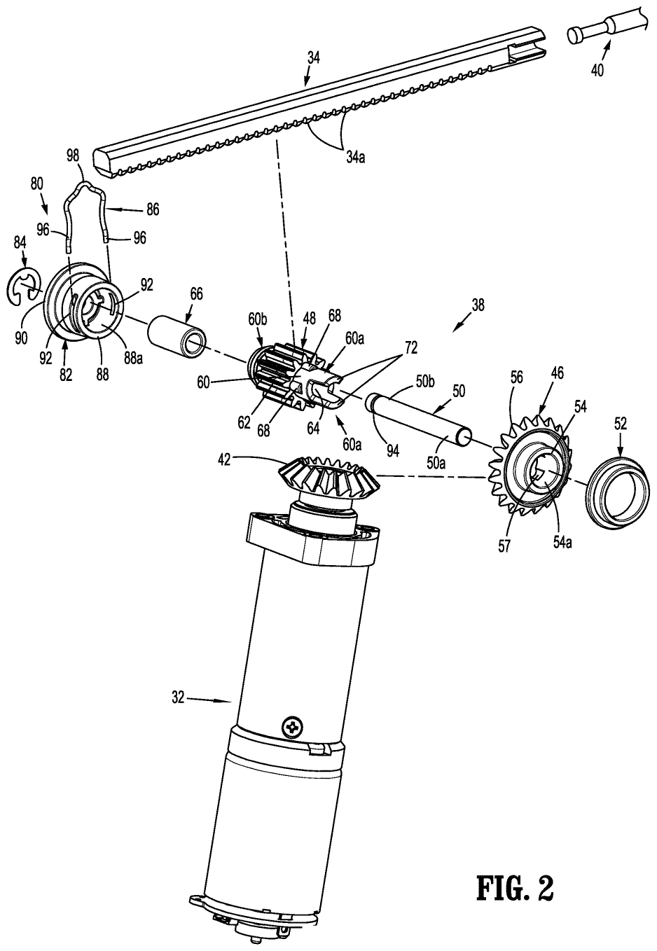

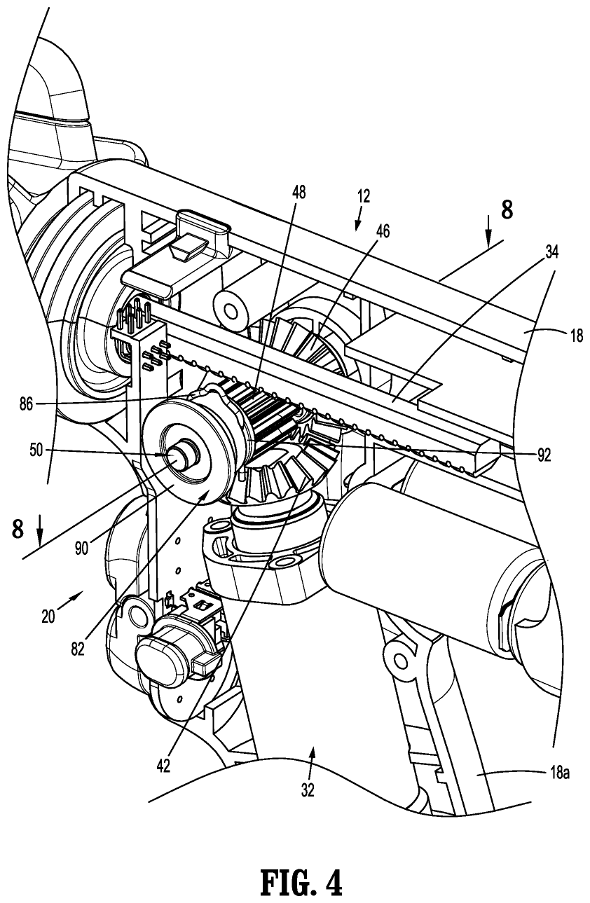

FIGS. 2-5 illustrate internal components of the handle assembly 12 (FIG. 1) which include a motor assembly 32, a toothed rack 34, a retainer assembly 80, a gear assembly 38, and a firing rod 40. The toothed rack 34 has a distal end that is coupled to a proximal end of the firing rod 40 such that longitudinal movement of the toothed rack 34 causes longitudinal movement of the firing rod 40. The motor assembly 32 includes a motor output gear 42 that can be in the form of a bevel gear. The gear assembly 38 is engaged with the motor output gear 42 to translate rotational movement of the motor output gear 42 to longitudinal movement of the toothed rack 34. In aspects of the disclosure, the stationery handle 18a of the housing 18 of the handle assembly 12 (FIG. 1) receives the motor assembly 32.

The gear assembly 38 includes a first gear 46, a second gear 48, and a gear shaft 50. In aspects of the disclosure, the first gear 46 is in the form of a bevel gear that is rotatably supported within the cavity 22 (FIG. 3) of the housing 18 of the handle assembly 12 on a bearing 52. The first gear 46 includes a central hub 54 and outer gear teeth 56 disposed about the hub 54. The hub 54 defines a central through bore 54a that receives the gear shaft 50. The hub 54 of the first gear 46 includes an inner surface that defines recesses 57. The outer gear teeth 56 of the first gear 46 are engaged with the motor output gear 42 such that rotation of the motor output gear 42 causes rotation of the first gear 46.

The gear shaft 50 extends transversely across the cavity 22 (FIG. 3) of the housing 18 of the handle assembly 12 (FIG. 1) and has a first end portion 50a and a second end portion 50b. The first end portion 50a of the gear shaft 50 extends through the first and second gears 46, 48 and the second end 50b of the gear shaft 50 is supported on an opposite side of the housing 12 as described in further detail below. The second gear 48 is rotatably supported about the gear shaft 50 and is engaged with rack teeth 34a of the toothed rack 34.

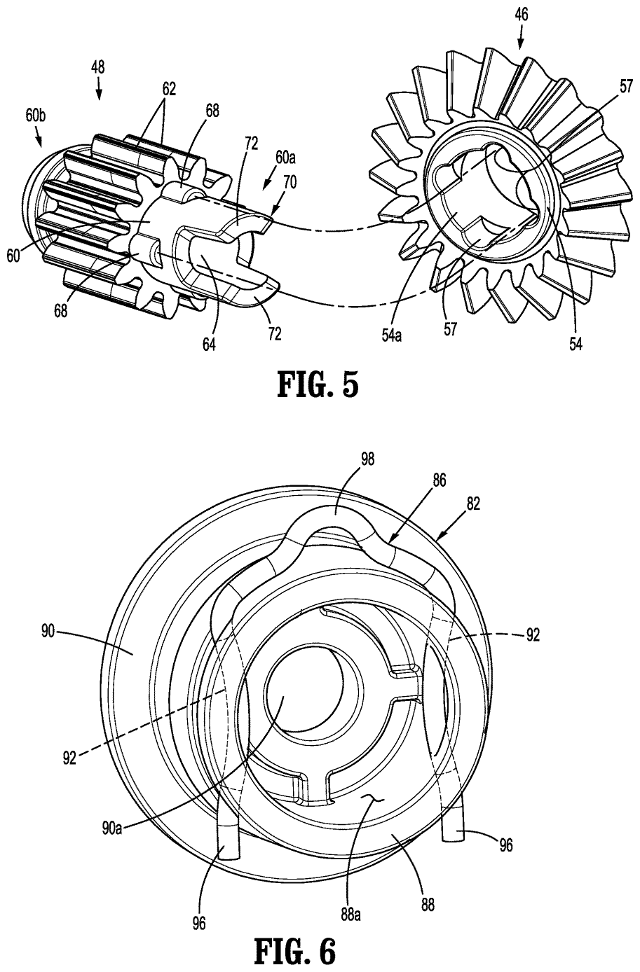

The second gear 48 includes a cylindrical hub 60 and outer gear teeth 62 that are positioned about the cylindrical hub 60. The cylindrical hub 60 of the second gear 48 defines a central through bore 64 that receives the gear shaft 50 such that the second gear 48 is rotatable about the gear shaft 50. The central through bore 64 of the second gear 48 is stepped and receives a cylindrical bearing 66 that rotatably supports the second gear 48 on the gear shaft 50. The cylindrical hub 60 of the second gear 48 has a first end portion 60a and a second end portion 60b. The first end portion 60a extends beyond the gear teeth 62 of the second gear 48 into the central through bore 54a of the first gear 46 and includes an outer surface that has one or more protrusions 68 (FIG. 5) that can received within the recesses 57 of the first gear 46 to secure the first gear 46 to the second gear 48. When the protrusions 68 are received in the recesses 56, the second gear 48 is secured to the first gear 46 such that rotation of the first gear 46 causes corresponding rotation of the second gear 48. The second gear 48 is movable along the gear shaft 50 between a first position in which the protrusions 68 of the second gear 48 are received within the recesses 57 of the first gear 46 and a second position in which the protrusions 68 are spaced from the recesses 57 of the first gear 46. In the second position of the second gear 48, the second gear 48 is rotatable about the gear shaft 50 independently of the first gear 46.

The first end portion 60a of the cylindrical hub 60 of the second gear 48 also includes a first key structure 70 that is configured to releasably engage a second key structure 112 formed on a retraction tool 110 of the stapling device 10. In aspects of the disclosure, the first key structure 70 includes castellations 72. The second end portion 60b (FIG. 2A) of the central hub portion 60 of the second gear 48 extends beyond the outer gear teeth 62 in a direction opposite to the first end portion 60a and includes an angled or tapered end surface 74 (FIG. 2A) and an annular groove 76 that is positioned between the tapered end surface 74 of the central hub 60 and the outer gear teeth 62.

FIGS. 2 and 6 illustrate the retainer assembly 80 which includes a retainer body 82, a shaft clip 84 (FIG. 2), and a gear clip 86. The retainer body 82 includes a cylindrical portion 88 and a flange 90 that extends about one end of the cylindrical portion 88. The cylindrical portion 88 of the retainer body 82 defines a through bore 88a and slots 92 that extend through the cylindrical portion 88 and communicate with the through bore 88a. The slots 92 are diametrically opposed to each other on the cylindrical portion 88 and receives the gear clip 86 as described in further detail below. The flange 90 of the retainer body 82 defines a through bore 90a (FIG. 6) that communicates with the through bore 88a of the cylindrical portion 88 and receives the second end 50b (FIG. 2) of the gear shaft 50 to support the second end 50b of the gear shaft 50. The second end 50b of the gear shaft 50b defines an annular slot 94 that receives the shaft clip 84 to secure the gear shaft 50 to the retainer body 82 of the retainer assembly 80.

The gear clip 86 is formed of a resilient material and includes legs 96 and a backspan 98. The legs 96 extend through the slots 92 in the retainer body 82 into the through bore 88a of the retainer body 82 to a position to engage the end of the second end portion 60b of the second gear 48 of the gear assembly 48 when the second gear 48 is in the first position (FIG. 8). When the second gear 48 is moved from the first position to the second position, the legs 96 of the gear clip 86 flex outwardly and pass over the tapered end surface 74 of the second gear 48 and snap into the annular groove 76 in the second end portion 60b of the second gear 48 to retain the second gear 48 in the second position.

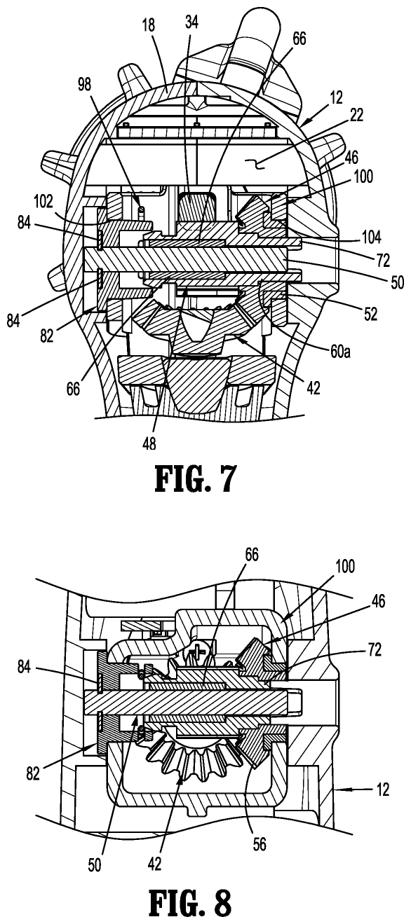

In aspects of the disclosure, an inner housing 100 (FIG. 7) is received and supported within the cavity 22 of the housing 18 of the handle assembly 12. The inner housing 100 defines a first opening 102 and a second opening 104. The first opening 102 of the inner housing 100 receives the cylindrical portion 88 of the retainer body of the 82 of the retainer assembly 80, and the second opening 104 receives the bearing 52 to rotatably support the first gear 46 of the gear assembly 38 on the inner housing 100. The second gear 48 is rotatably supported within the central through bore 54a of the first gear 46 and the first end portion 50a of the gear shaft 50 is received within the central through bore 64 of the second gear 48.

FIGS. 7 and 8 illustrate internal components of the handle assembly 12 of the stapling device 10 (FIG. 1) with the second gear 48 of the gear assembly 38 in the first position. When the second gear 48 is the first position, the first end portion 60a of the second gear 48 is received within the central through bore 54a of the first gear 46 such that the protrusions 68 on the first end portion 60a are received in the recesses 57 of the first gear 46. When the motor assembly 32 (FIG. 2) is activated by pressing the actuation button or buttons 20 (FIG. 1), the motor output gear 42 which is engaged with the first gear 46 of the gear assembly 38 rotates the first gear 46. Since the second gear 48 of the gear assembly 38 is in the first position with the protrusions 68 of the second gear 48 received within the recesses 57 of the first gear 46, the second gear 48 rotates with the first gear 46 about the gear shaft 50. The gear teeth 62 of the second gear 48 are engaged with teeth 34a of the toothed rack 34 (FIG. 2) such that rotation of the second gear advances the toothed rack 34 to advance or retract the firing rod 40 (FIG. 2).

In aspects of the disclosure, the tool assembly 16 (FIG. 1) includes a cartridge assembly 106 and an anvil 108, and the firing rod 40 (FIG. 2) is coupled to a clamp member (not shown) that is movable through the tool assembly 16 to move the tool assembly 16 between an unclamped position and a clamped position and to subsequently eject staples from the cartridge assembly 106 into the anvil 108. It is envisioned that the tool assembly 16 can be configured to perform other surgical functions other than stapling such as vessel sealing, suturing, or the like.

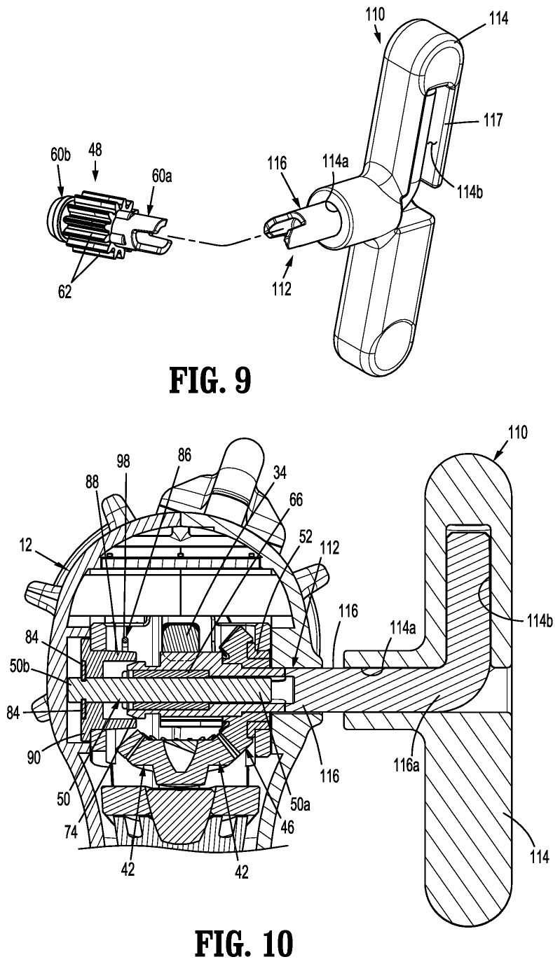

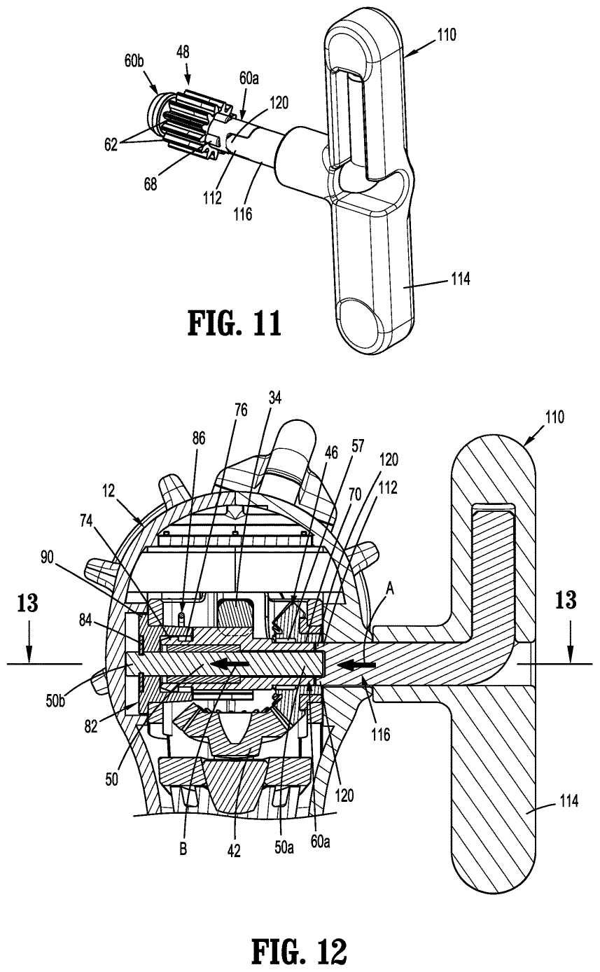

FIGS. 9 and 10 illustrate the retraction tool 110 which includes a handle 114 and an operating member 116 that extends from the handle 114. In aspects of the disclosure, the retraction tool 110 has a T-shaped configuration and the operating member 116 extends from the handle 114 and supports the second key structure 112. The handle 114 of the retraction tool 110 defines a circular bore 114a and an open slot 114b and the operating member 116 has an L-shaped body 116a that has a portion 116b that is received within the received within the slot 114b and a portion that extends through circular bore 114a. The slot 114b defines an opening 117 on one side of the slot 114a. The opening 117 prevents translation of rotatable movement of the handle 114 to rotational movement of the operating member 116 when the handle 114 is rotated in a direction that removes the operating member 116 from the slot 114a, i.e., when the handle 114 of the retraction tool 110 is rotated in a direction that would effect advancement of the toothed rack 34, the handle 114 rotates independently of the operating member 116 such that the portion of the operating member 116 received within the slot 114b rotates from within the slot 114a to a position outside of the slot 114b. Alternately, the retraction tool 110 can have a variety of configurations that allow for retraction and/or advancement of the toothed rack 34.

The operating member 116 of the retraction tool 110 is dimensioned to be received through an access port 118 (FIG. 1) formed in the housing 18 of the handle assembly 12. The access port 118 is positioned adjacent to the first end portion 60a of the second gear 48 such that the second key structure 112 of the operating member 116 of the retraction tool 110 engages the first key structure 70 of the second gear 48 when the operating member 116 is inserted through the access port 118. In aspects of the disclosure, the second key structure 112 includes castellations 120 that mesh with the castellations 72 of the first key structure 70 of the second gear 48 when the operating member 116 is inserted through the access port 118.

FIGS. 12-14 illustrate the retraction tool 110 inserted in the direction indicated by arrow “A” into the access port 118 of the housing 18 of the handle assembly 12 to manually retract the toothed rack 34. When the retraction tool 110 is inserted into the access port 118 of the housing 18 of the handle assembly 12 (FIG. 1), the operating member 116 of the retraction tool 110 engages the first end portion 60a of the second gear 48 such that the second key structure 112 of the retraction tool 110 meshes with the first key structure 70 of the second gear 48. As the retraction tool 110 is pushed further through the access port 118 in the housing 18 of the tool assembly 12, the operating member 116 of the retraction tool 110 pushes the second gear 48 in the direction of arrow “B” along the gear shaft 50 from the first position to the second position to remove the protrusions 68 of the second gear 48 from the recesses 57 of the first gear 46 and disengage the second gear 48 from the first gear 46.

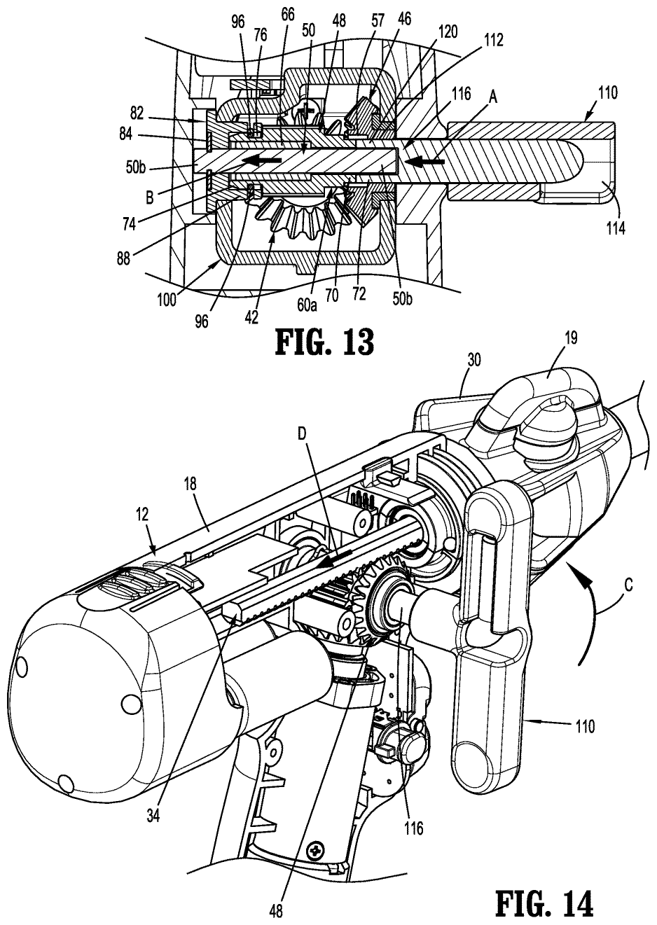

As the second gear 48 moves in the direction of arrow “B” in FIG. 13, the tapered end surface 74 of the second end portion 60b of the second gear 48 engages the legs 96 of the gear clip 86 and deforms the legs 96 outwardly to allow the second end portion 60b of the second gear 48 to move past the legs 96. When the second end portion 60b of the second gear 48 moves to a position in which the legs 96 of the gear clip 86 are aligned with the annular groove 76 in the second end portion 60b of the second gear 48, the legs 96 of the gear clip 86 snap into the annular groove 76 to retain the second gear 48 in the second position.

Once the gear clip 86 of the retainer assembly 80 is received within the annular groove 76 of the second gear 48, the second gear 48 is retained in the second position. In the second position, the second gear 48 is engaged with the toothed rack 34 but disengaged from the first gear 46. When the retraction tool 110 is rotated in the direction indicated by arrow “C” in FIG. 14, the second gear 48 which is engaged with the operating member 116 of the retraction tool 110 rotates about the gear shaft 50 to move the toothed rack 34 in the direction indicated by arrow “D” in FIG. 14 to retract the firing rod 40 (FIG. 2). As described above, the firing rod 40 is engaged with a clamp member (not shown) of the tool assembly 16 (FIG. 1) that is movable through the tool assembly 16 to actuate the tool assembly 16.

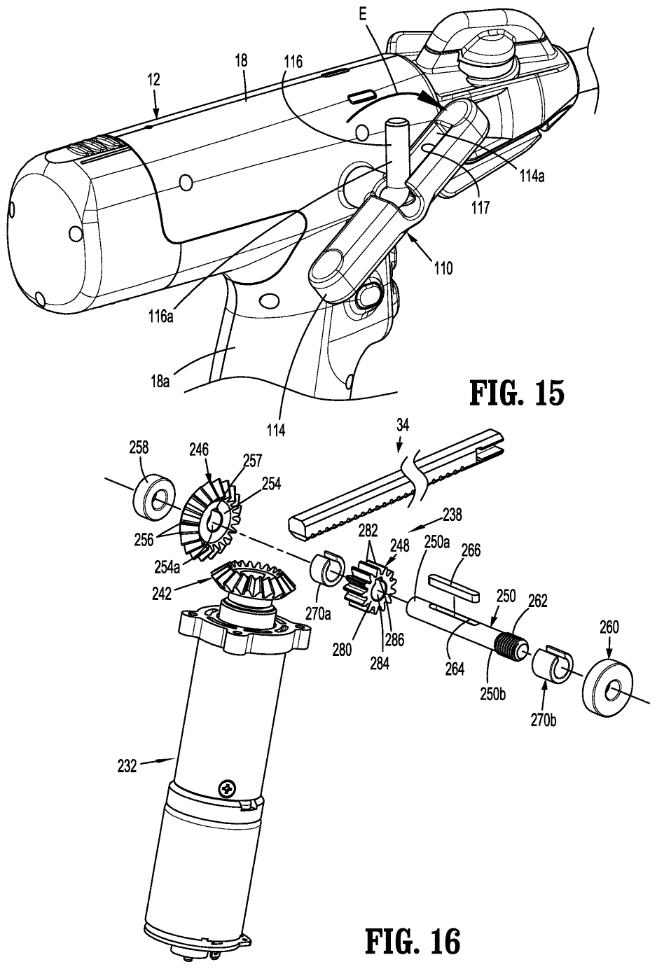

FIG. 15 illustrates rotation of the retraction tool 110 in a direction indicated by the arrow “E” that would cause the toothed rack 34 (FIG. 2) to advance. When the retraction tool 110 is rotated in the direction of arrow “E”, the handle 114 of the retraction tool 110 rotates independently of the body 116a of the operating member 116. More specifically, since the operating member 116 is not confined within the slot 114a of the handle 114, the body 116a of the operating member 116 moves through the opening 117 in the handle 114 and exits the slot 114a when an attempt is made to advance the toothed rack 34. As such, the second gear 48 (FIG. 14) will not rotate in a direction to advance the toothed rack 34 because the operating member 116 does not rotate with the handle 114 of the retraction tool 110.

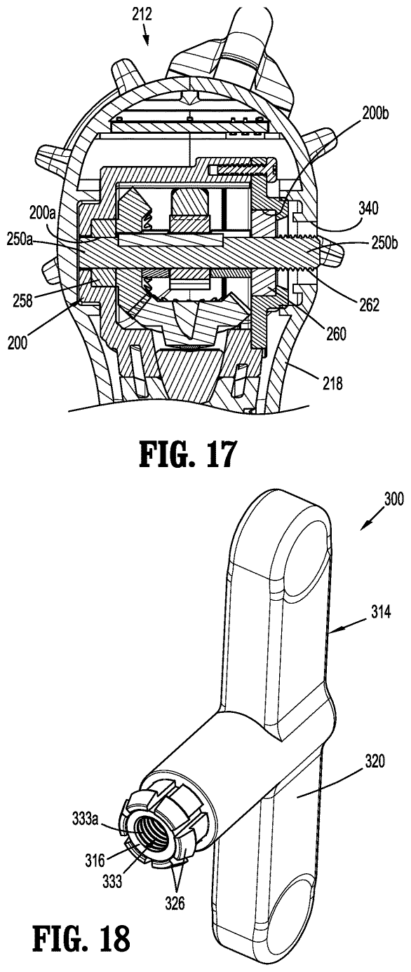

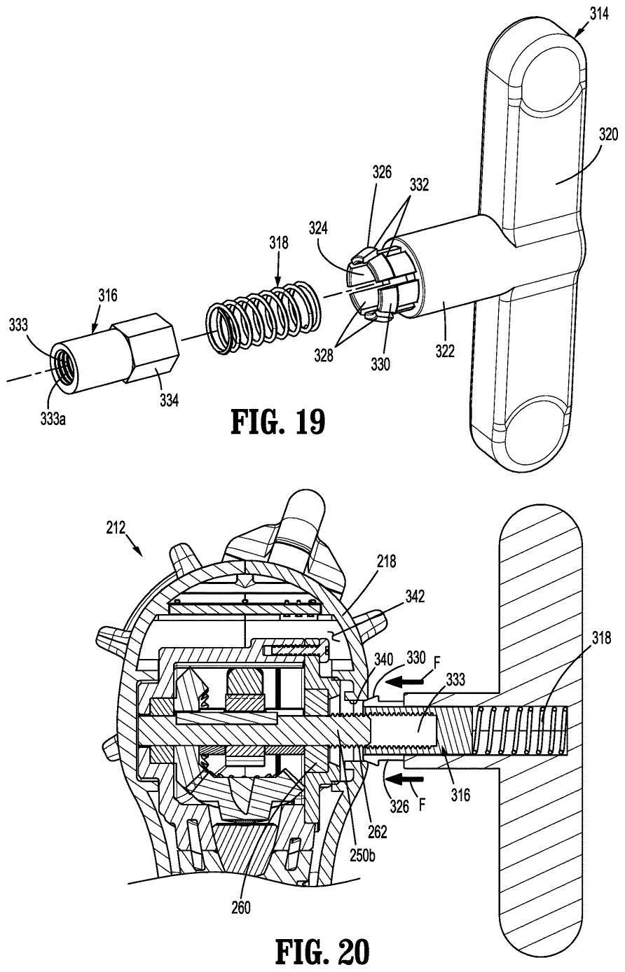

FIGS. 16-25 illustrate an alternate version of the handle assembly 12 of the stapling device shown in FIG. 1 shown generally as handle assembly 212 (FIG. 17). FIGS. 16 and 17 illustrate the handle assembly 212 which includes a housing 218 and a gear assembly 238 which is illustrated in and includes a first gear 246, a second gear 248, and a gear shaft 250. The first gear 246 is rotatably supported within the housing 218 (FIG. 1) of the handle assembly 212 and includes a central hub 254 and outer gear teeth 256 disposed about the central hub 254. The central hub 254 of the first gear 246 defines a through bore 254a that receives the gear shaft 250 and includes an inner surface that defines a first notch 257. The outer gear teeth 256 of the first gear 246 are engaged with the motor output gear 242 of the motor assembly 232 such that rotation of the motor output gear 242 causes rotation of the first gear 246.

The gear shaft 250 extends transversely across the cavity 22 (FIG. 3) of the housing 18 of the handle assembly 12 (FIG. 1) and has a first end portion 250a and a second end portion 250b. The first end portion 250a of the gear shaft 250a is rotatably supported within a first bearing 258 that is supported within a first recess 200a defined in the inner housing 200 of the handle assembly 212 (FIG. 1). The second end portion 250b of the gear shaft 250 is supported within a second bearing 260 and includes screw threads 262. The second bearing 260 is supported within a second recess 200b defined in the inner housing 200 of the handle assembly 212 (FIG. 1).

The gear shaft 250 defines an elongate slot 264 that receives a key 266 that is received within the elongate slot 264 and projects outwardly from the gear shaft 250. The gear shaft 250 is longitudinally movable within the first and second bearings 258 and 260 between first and second positions. In the first position, the key 266 is received within the first notch 257 of the first gear 246 such that rotation of the first gear 246 causes corresponding rotation of the gear shaft 250.

The second gear 248 is supported on the gear shaft 250 between the first bearing 258 and the second bearing 260 and is maintained in spaced relation to the first gear 246 and the second bearing 260 by spacers 270a and 270b, respectively. The second gear 248 includes a cylindrical hub 280 and outer gear teeth 282 that are positioned about the cylindrical hub 280. The cylindrical hub 280 of the second gear 248 defines a central through bore 284 that receives the gear shaft 250 and defines a second notch 286. The key 266 is secured to the gear shaft 250 and is received within the second notch 286 of the second gear 248 such that rotation of the gear shaft 250 causes rotation of the second gear 248.

FIGS. 18-20 illustrate the retraction tool 300 which includes a handle 314, an operating member 316, and a biasing member 318. The handle 314 includes a grip portion 320 that defines a longitudinal axis and a hollow body portion 322 that extends in a direction that is transverse to the longitudinal axis defined by the grip portion 320. The hollow body portion 322 defines a bore 324 and has a first end that is secured to, or formed integrally with, the grip portion 320 and a second end that supports a retainer 326. The retainer 326 includes a plurality of resilient fingers 328 that extend about the bore 324 of the hollow body portion 322 of the handle 314. Each of the resilient fingers 328 has a tapered or angled end 330 that defines an outwardly facing shoulder 332 that is positioned between the tapered end 330 and the hollow body portion 322.

The operating member 316 is non-rotatably received within the bore 324 of the hollow body portion 322 of the handle 314. In aspects of the disclosure, the operating member 316 defines an internal bore 333 that includes internal threads 333a and includes a non-circular head 334, e.g. a hexagonal head, and the bore 324 has a corresponding shape to prevent rotation of the operating member 316 within the bore 324. The biasing member 318 is positioned within the bore 324 of the hollow body portion 322 between the operating member 316 and the grip portion 320 to urge the operating member 316 towards resilient fingers 328 and an open end of the bore 324.

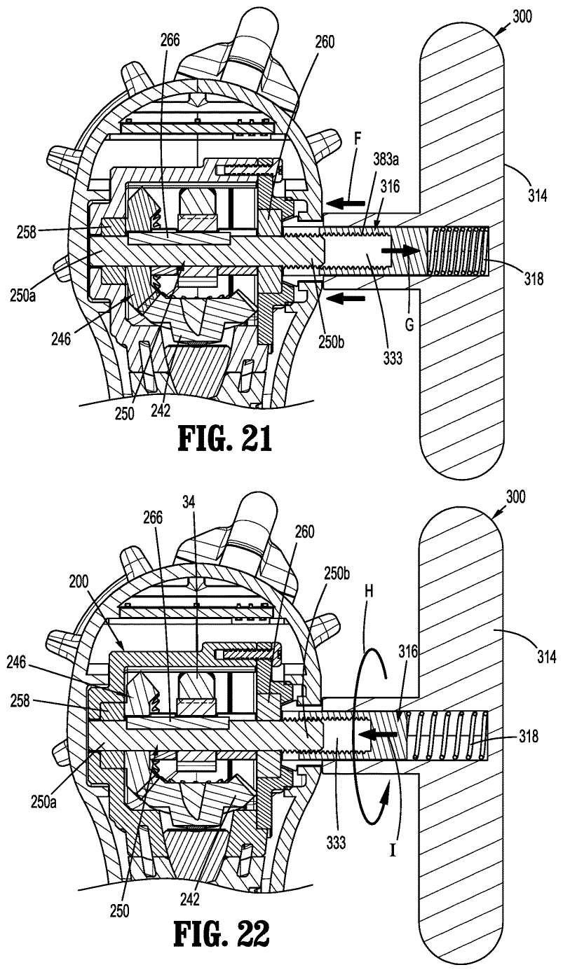

FIGS. 20 and 21 illustrate the retraction tool 300 as the retraction tool 300 is inserted into the access port 340 of the housing 218 of the handle assembly 212 in the direction indicated by arrow “F”. When the operating member 316 of the retraction tool 300 is inserted through the access port 340 of the housing 218 of the handle assembly 212, the operating member 316 is urged into engagement with the second end portion 250b of the gear shaft 250 by the biasing member 318 such that the second end portion 250b of the gear shaft 250 is received within the bore 333 of the operation member 316. When the second end portion 250b of the gear shaft 250 is received within the bore 333 of the operating member 316, the screw threads 262 of the gear shaft 250 engage the internal threads 333 of the operating member 316. This engagement is maintained by the biasing member 318 which compresses upon engagement of the operating member 316 with the gear shaft 250.

As the operating member 316 moves through the access port 340 of the housing 218, the tapered ends 330 of the resilient fingers 328 of the retainer 326 engage inner surfaces of the housing 318 defining the access port 340 and flex inwardly as the resilient fingers 328 pass through the access port 340. As this occurs, the operating member 316 is urged in the direction of arrow “G” further into the bore 324 of the handle 314 in the direction indicated by arrow “G” in FIG. 21 to compress the biasing member 318. When the shoulders 332 of the resilient fingers 328 pass into the cavity 342 defined by the housing 218, the resilient fingers 328 snap outwardly such that the shoulders 332 of the resilient fingers 328 engage an internal wall of the housing 318 to secure the retraction tool 300 to the housing 218 of the handle assembly 212 (FIG. 21).

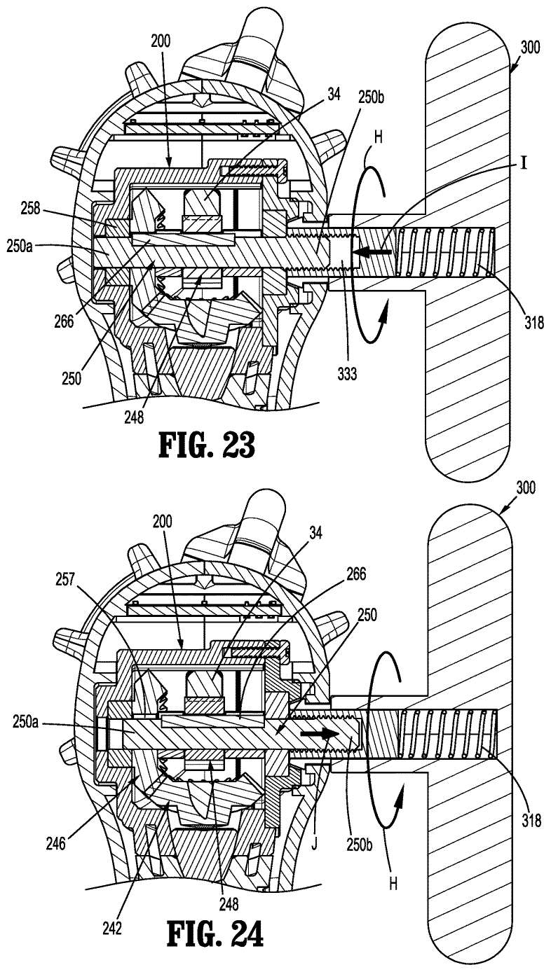

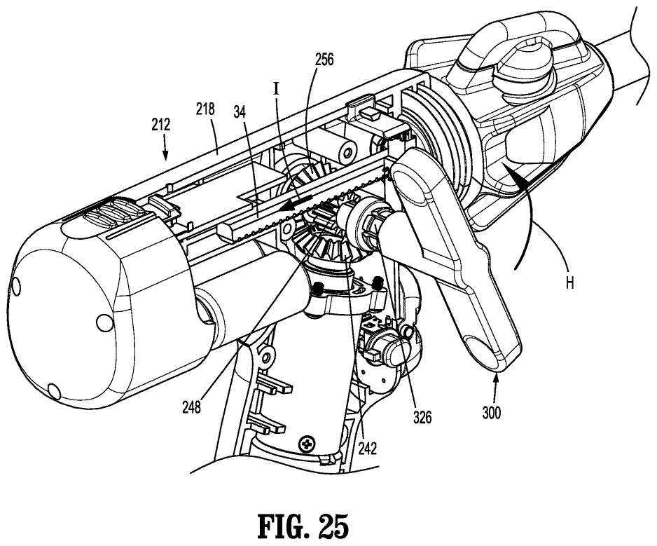

FIGS. 22 and 23 illustrate the handle assembly 12 with the retraction 300 retained within the access port 340 as the gear shaft 250 is moved from a first position engaged with the first gear 246 to a second position disengaged from the first gear 246. Once the retraction tool 300 is secured to the housing 218 of the handle assembly 212 by the retainer 326 and the retraction tool 300 is rotated in the direction indicated by arrow “H” in FIG. 22, the internal threads 333a of the operating member 316 engage the threads 262 of the gear shaft 250. When the retraction tool 300 is rotated in the direction of arrow “H” in FIGS. 22 and 23, the operating member 316 moves along the gear shaft 250 in the direction indicated by arrow “I” into the cavity 342 of the housing 218 of the handle assembly 212. When the operating member 316 engages the bearing 260 (FIG. 24) and cannot moves any further into the housing 218 of the handle assembly 212, the gear shaft 250 is pulled in the direction indicted by arrow “J” into the bore 333 of the operating member 316 to move the gear shaft 250 from the first position to the second position. As described above, when the gear shaft 250 moves to the second position, the key 266 that is secured within the elongate slot 264 of the gear shaft 250 is withdrawn from the first notch 257 (FIG. 16) to disengage the first gear 246 from the gear shaft 250.

FIGS. 24 and 25 illustrate the retraction tool 300 after the retraction tool 300 is rotated sufficiently to cause the second end portion 250b of the gear shaft 250 to bottom out in the bore 333 of the operating member 316 of the retraction tool 300. Once the gear shaft 250 bottoms out in the bore 333 of the operating member 316 of the retraction tool 300, further rotation of the retraction tool 300 in the direction of arrow “H” rotates the gear shaft 250 to cause rotation of the second gear 248. When the second gear 248 is rotated in the direction of arrow “H”, the toothed rack 34 is retracted in the direction of arrow “I”.

It is noted that because of the threaded engagement between the threaded operating member 316 of the retraction tool 300 and the threaded gear shaft 250, rotation of the retraction tool 300 will not function to advance the toothed rack 34. More specifically, if the retraction tool 300 were to be rotated in a direction opposite to the direction indicated by arrow “H” in FIG. 25, the operating member 316 of the retraction tool 300 will disengage, i.e., unthread, from the gear shaft 250. As such, the retraction tool 300 will not function to advance the toothed rack 34. Alternately, the retraction tool can have a variety of configurations that allow for retraction and/or advancement of the toothed rack 34.

Persons skilled in the art will understand that the devices and methods specifically described herein and illustrated in the accompanying drawings are non-limiting exemplary aspects of the disclosure. It is envisioned that the elements and features illustrated or described in connection with one exemplary aspect of the disclosure may be combined with the elements and features of another without departing from the scope of the disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. A powered handle assembly for a surgical device comprising:
a housing defining a cavity and an access opening that communicates with the cavity;
a motor assembly supported within the cavity of the housing and including a motor shaft and an output gear secured to the motor shaft;
a toothed rack received within the cavity of the housing, the toothed rack supported for longitudinal movement between a rack retracted position and a rack advanced position;
a gear shaft supported within the cavity of the housing;
a first gear rotatably supported on the gear shaft, the first gear engaged with the output gear to translate rotational movement of the output gear to rotation of the first gear;
a second gear rotatably supported on the gear shaft and engaged with the toothed rack such that rotation of the second gear causes longitudinal movement of the toothed rack, the second gear linearly movable along the gear shaft from a first position in which the second gear is coupled to the first gear such that rotation of the first gear causes rotation of the second gear, to a second position in which the second gear can rotate independently of the first gear; and
a retraction tool including a handle and an operating member, the operating member insertable through the access opening in the housing into engagement with the second gear to an engaged position, wherein in the engaged position, the second gear is moved from the first position to the second position and the operating member is engaged with the second gear such that rotation of the operating member causes rotation of the second gear.

2. The powered handle assembly of claim 1, further including a retainer assembly supported within the housing, the retainer assembly configured to engage the second gear to retain the second gear in the second position.

3. The powered handle assembly of claim 2, wherein the second gear includes a first end portion including protrusions, and the first gear includes a central hub defining a through bore, an inner surface of the central hub defining recesses that receive the protrusions of the second gear when the second gear is in the first position.

4. The powered handle assembly of claim 3, wherein the second gear includes a second end portion defining an annular groove, and the retainer assembly includes a gear clip having resilient legs that are received within the annular groove when the second gear is in the second position to retain the second gear in the second position.

5. The powered handle assembly of claim 4, wherein the second end portion of the second gear includes a tapered surface, the tapered surface biasing the resilient legs outwardly to allow the resilient legs to move into the annular groove as the second gear moves from the first position to the second position.

6. The powered handle assembly of claim 3, wherein the operating member of the retraction tool includes first castellations and the first end portion of the second gear includes second castellations, the first castellations meshing with the second castellations to couple the operating member of the retraction tool to the second gear such that rotation of the operating member of the retraction tool causes rotation of the second gear.

7. The powered handle assembly of claim 6, wherein the retraction tool includes a handle and the operating member, the handle coupled to the operating member such that rotation of the handle in a first direction causes corresponding rotation of the operating member and rotation of the handle in a second opposite direction causes the handle to disengage from the operating member.

8. The powered handle assembly of claim 7, wherein the handle has a T-shaped configuration and defines a slot having an open side and a circular bore, the circular bore defining a longitudinal axis that is perpendicular to a longitudinal axis defined by the slot, and the operating member is L-shaped and includes a first portion received within the circular bore and a second portion received within the slot such that rotation of the handle of the retraction tool in the second opposite direction causes the operating member to exit from the open side of the slot.

9. The powered handle assembly of claim 8, wherein rotation of the handle of the retraction tool in the first direction causes the toothed rack to move towards the rack retracted position.

10. A retraction tool comprising:
a handle having a T-shaped configuration and defining a slot having an open side and a circular bore, the circular bore defining a longitudinal axis that is perpendicular to a longitudinal axis defined by the slot; and
an operating member having an L-shaped configuration including a first portion received within the circular bore and a second portion received within the slot, wherein rotation of the handle in a first direction causes corresponding rotation of the operating member and rotation of the handle in a second opposite direction causes the second portion of the operating member to exit from the slot via the open side of the slot.

11. A surgical stapling device comprising:
a handle assembly including:
a housing defining a cavity and an access opening that communicates with the cavity;
a motor assembly supported within the cavity of the housing and including a motor shaft and an output gear secured to the motor shaft;
a toothed rack received within the cavity of the housing, the toothed rack supported for longitudinal movement between a rack retracted position and a rack advanced position;
a gear shaft supported within the cavity of the housing;
a first gear rotatably supported on the gear shaft, the first gear engaged with the output gear to translate rotational movement of the output gear to rotation of the first gear;
a second gear rotatably supported on the gear shaft and engaged with the toothed rack such that rotation of the second gear causes longitudinal movement of the toothed rack, the second gear linearly movable along the gear shaft from a first position in which the second gear is coupled to the first gear such that rotation of the first gear causes rotation of the second gear, to a second position in which the second gear can rotate independently of the first gear; and
a retraction tool including a handle and an operating member, the operating member insertable through the access opening in the housing into engagement with the second gear to an engaged position, wherein in the engaged position, the second gear is moved from the first position to the second position and the operating member is engaged with the second gear such that rotation of the operating member in a first direction causes rotation of the second gear;
an adapter assembly having a proximal portion and a distal portion, the proximal portion coupled to the handle assembly;
a firing rod having a proximal portion coupled to the toothed rack and a distal portion; and
a tool assembly supported on the distal portion of the adapter assembly, the tool assembly operatively associated with the firing member.

12. The surgical stapling device of claim 11, further including a retainer assembly supported within the housing, the retainer assembly configured to engage the second gear to retain the second gear in the second position.

13. The surgical stapling device of claim 12, wherein the second gear includes a first end portion including protrusions, and the first gear includes a central hub defining a through bore, an inner surface of the central hub defining recesses that receive the protrusions of the second gear when the second gear is in the first position.

14. The surgical stapling device of claim 13, wherein the second gear includes a second end portion defining an annular groove, and the retainer assembly includes a gear clip having resilient legs that are received within the annular groove when the second gear is in the second position to retain the second gear in the second position.

15. The surgical stapling device of claim 13, wherein the second end portion of the second gear includes a tapered surface, the tapered surface biasing the resilient legs outwardly to allow the resilient legs to move into the annular groove as the second gear moves from the first position to the second position.

16. The surgical stapling device of claim 13, wherein the operating member of the retraction tool includes first castellations and the first end portion of the second gear includes second castellations, the first castellations meshing with the second castellations to couple the operating member of the retraction tool to the second gear such that rotation of the operating member of the retraction tool causes rotation of the second gear.

17. The surgical stapling device of claim 16, wherein the retraction tool includes a handle and the operating member, the handle coupled to the operating member such that rotation of the handle in a first direction causes corresponding rotation of the operating member and rotation of the handle in a second opposite direction causes the handle to disengage from the operating member.

18. The surgical stapling device of claim 17, wherein the handle has a T-shaped configuration and defines a slot having an open side and a circular bore, the circular bore defining a longitudinal axis that is perpendicular to a longitudinal axis defined by the slot, and the operating member is L-shaped and includes a first portion received within the circular bore and a second portion received within the slot such that rotation of the handle of the retraction tool in the second opposite direction causes the operating member to exit from the open side of the slot.

19. The surgical stapling device of claim 11, wherein the tool assembly includes an anvil and a cartridge assembly.

20. The surgical stapling device of claim 17, wherein rotation of the handle of the retraction tool in the first direction causes the toothed rack to move towards the rack retracted position.
